(19) United States
(12) Patent Application Publication
(10) Pub. No.: US 2005/0129666 A1
(43) Pub. Date: Jun. 16, 2005

(54) Compositions comprising preconditioned myoblasts having enhanced fusion properties

(75) Inventor: Jacques P. Tremblay, Quebec (CA)

Correspondence Address: BIRCH STEWART KOLASCH & BIRCH, PO BOX 747, FALLS CHURCH, VA, 22040-0747, US

(73) Assignee: Universite Laval

(21) Appl. No.: 10/936,823

(22) Filed: Sep. 9, 2004

Related U.S. Application Data
(63) Continuation of application No. 10/058,281, filed on Jan. 30, 2002, now abandoned.
Application No. 10/058,281 is a continuation-in-part of application No. 09/188,413, filed on Nov. 10, 1998, now abandoned.
Application No. 09/188,413 is a continuation-in-part of application No. 08/404,888, filed on Mar. 16, 1995, now Pat. No. 5,833,978.

(51) Int. Cl. A61K 45/00; C12N 5/08

(52) U.S. Cl. 424/93.7; 435/366

(57) ABSTRACT

The present invention relates to a method for preconditioning healthy donor's myoblasts in vitro before transplantation thereof in compatible patients suffering of recessive myopathies, particularly of muscular dystrophy. This in vitro preconditioning improves the success of the transplantation while not requiring an in vivo preconditioning of the patient's muscle by irradiation or by administering muscular toxin. The invention further relates to compositions comprising such preconditioned myoblasts. The preconditioning comprises pre-treating the transplanted myoblasts with human fibroblast growth factor (bFGF). The transplantation is made with the whole culture along with bFGF. A concentration of 100 ng/ml bFGF improved the myoblasts fusion by a four fold average.

### RELATED U.S. APPLICATION DATA

Continuation-in-part of U.S. Ser. No. 188,413 filed Nov. 11, 1998 which is a continuation-in-part of U.S. Ser. No. 404,888, Mar. 16, 1995, issued as U.S. Pat. No. 5,833,978.

### FIELD OF THE INVENTION

The present invention relates to a method for preconditioning healthy donor's myoblasts in vitro before transplantation thereof in compatible patients, particularly those suffering of recessive myopathies such as muscular dystrophy. This in vitro preconditioning improves the success of the transplantation while not requiring an in vivo preconditioning of the patient's muscle by irradiation or by administering muscular toxin. The invention further relates to compositions comprising such preconditioned myoblasts.

### BACKGROUND OF THE INVENTION

Duchenne muscular dystrophy (DMD) is a progressive disease characterized by the lack of dystrophin under the sarcolemmal membrane^{6,19,28,37}. One possible way to introduce dystrophin in the muscle fibers of the patients to limit the degeneration is to transplant myoblasts obtained from normal subjects^{30,34,35}. Several groups have tried myoblast transplantations to DMD patients but poor graft success was observed^{17,22,24,38}. Even in experimental myoblast transplantation using mdx mice, an animal model of DMD^{10,25,29}, large amount of dystrophin-positive fibers were observed only when nude mdx mice were previously irradiated to prevent regeneration of the muscle fibers by host myoblasts^{32,43}. High percentage of dystrophin-positive fibers was also observed in mdx mice immunosuppressed with FK 506 and in SCID mice, in both cases muscles were previously damaged by notexin injection and irradiated^{23,27}. These results indicate that to obtain successful myoblast transplantation, it is necessary to have not only an immunodeficient mouse or a mouse adequately immunosuppressed but also a host muscle which has been adequately preconditioned. It is, however, impossible in clinical studies to use damaging treatments such as marcaine, notexin and irradiation. If good myoblast transplantation results can be obtained without using such techniques, this would be very helpful for myoblast transplantation in humans.

Recently there has been an increasing interest on the effects of basic fibroblast growth factor (bFGF) and other growth factors on myoblast cultures and myoblast cell lines^{1,4,5}. Basic FGF has been reported to both stimulate proliferation and inhibit differentiation of skeletal myoblasts in vitro^{15,16}. Other growth or trophic factors like insulin growth factor 1, transferrin, platelet-derived growth factor, epidermal growth factor, adrenocorticotrophin and macrophage colony-stimulating factor as well as C kinase proteins activators or agonists by which the effect of bFGF is mediated²⁰ may also have similar or even better effects than bFGF on the success of myoblast transplantation⁷. The use of these stimulating properties to enhance the success of transplantation by in vitro preconditioning of donor's cells and to replace at least partially the use of previously known methods of in vivo preconditioning of recipients' cells has never been suggested before.

These thus remains a need to provide methods of preconditioning of myoblasts which enhance their muscle-fusion properties and to provide compositions comprising such preconditioned myoblasts.

The present invention seeks to meet these and other needs.

The present description refers to a number of documents, the content of which is herein incorporated by reference.

### SUMMARY OF THE INVENTION

The present invention relates to a method of in vitro preconditioning of myoblasts prior to their transplantation in patients, namely those affected by recessive myopathies, particularly by Duchenne muscular dystrophy (DMD). In a DMD animal model (mdx), compatible donor mouse myoblasts were grown in culture with muscular growth or trophic factors, particularly, human basic Fibroblast Growth Factor (bFGF), before transplanting them in muscles of mdx mice without any previous damaging treatment. A four fold increase in the percentage of muscle fibers expressing dystrophin, which is indicative of functional muscle cells, was obtained with pretreatment with bFGF. These experimental results are expected to verify in naturally occurring dystrophy or other types of recessive myopathies in animal and human subjects, since the mdx mouse is an animal model wherein muscular dystrophy is naturally occurring. In such a case, human myoblasts are to be used preferably and to be treated with bFGF prior to transplantation.

The present invention further relates to compositions comprising preconditioned myoblasts having enhanced fusion properties. More specifically, the invention relates to a composition comprising a culture of myoblasts having been preconditioned to fuse to recipient muscle cells by the action of at least one trophic factor, including basic fibroblast growth factor (bFGF). In a particular embodiment, the bFGF is supplied exogenously. In another particular embodiment, the bFGF is supplied endogenously after the bFGF gene sequence having been introduced into the myoblasts by genetic engineering.

In a particular embodiment, the myoblasts have been transfected with an expression vector expressing recombinant bFGF capable of producing bFGF in sufficient amounts to improve the fusion of the myoblasts upon transplantation into a recipient individual over and above the fusion of the same number of myoblasts not producing this amount of bFGF. This sufficient amount is a “muscle fusion promoting amount”. Although an amount of 100 ng/ml (added exogenously) has been shown to produce a four fold increase in muscle cell fusion, this increase is an average as seen from Table 1. The increase is from about two to twenty fold with an exogenous dose of 100 ng/ml bFGF. Concentrations of 10 ng to 1 μg bFGF per ml of composition are within the scope of this invention, as concentrations capable of increasing by at least two fold the fusion of myoblasts.

The present invention further relates to methods of screening for agents which modulate the fusion properties of the myoblasts comprising an incubation of a composition of the present invention in the presence of an agent, and an assessment of the fusion properties of the myoblasts treated with the agent in comparison with a control composition (lacking this agent). A positive control would be bFGF.

While the preconditioning has been shown in the present disclosure to be produced by the addition of bFGF to the culture medium (exogenously added bFGF). The present invention should not be so limited. Indeed, a conditioning of the myoblasts may also be produced by having the myoblasts to endogenously produced bFGF into the culture (i.e. through transfections and the like). This can be done by introducing the whole human bFGF gene (Genbank accession numbers J04513 and E02544) or the bFGF cDNA in the cultured myoblasts. The genetically modified myoblasts will then secrete the bFGF factor in the culture medium in amounts sufficient to promote muscle fusion upon transplantation. The resulting presence of bFGF will precondition the myoblasts for successful transplantation, because the myoblasts will be grown in the presence of bFGF and transplanted therewith. The levels of bFGF to be reached in the culture for the purpose of this invention will comprise preferably between 10 ng/ml and 1 μg/ml. Such levels can be attained with genetic constructs having strong or inducible promoters. Of course, it is also within the scope of the present invention to provide bFGF (and/or other trophic factors) exogenously and endogenously.

The preconditioning effect may also be obtained by introducing into the myoblasts, a fragment of the whole gene or cDNA encoding the active segment of the bFGF protein. Of course, although human recombinant bFGF is preferred, other mammalian bFGF sequences can be used, provided that they retain their biological activity in enhancing the muscle fusion properties of the myoblasts. A non limiting example of such recombinant bFGF includes mouse bFGF.

In a preferred embodiment, the bFGF will be secreted by genetically engineered myoblasts enabling a preconditioning of the myoblasts. In a certain embodiment, mixed cultures of genetically engineered myoblasts and non-genetically engineered myoblasts can be used. In such an embodiment, the secretion of the bFGF would also precondition the non-genetically engineered myoblasts.

The complete bFGF gene, the bFGF cDNA or a fragment of the bFGF gene should be placed under the control of an adequate promoter to be expressed into the myoblasts. Such a promoter can be a viral promoter such as for example the SV40 promoter, the CMV promoter or a LTR promoter. The promoter controlling the expression of bFGF can also be that of a gene expressed in myoblasts, for example the promoter of desmin or actin or any other proteins expressed in myoblasts. The promoter may also be an inducible promoter, non limiting examples thereof include promoters which can be induced by tetracycline, cytokines, by modified hormones or by modified steroids.

The present invention also relates to a method of preconditioning myoblasts comprising a culturing of genetically engineered myoblasts expressing bFGF, a variant or a derivative thereof, having retained the fusion muscle enhancing properties of bFGF. The invention also relates to methods for improving the fusion of myoblasts and to methods of myoblasts transplantation comprising a culturing of the genetically engineered myoblasts of the present invention, and transplanting same into a recipient muscle tissue.

The treatment of the host should preferably include an adequate immunosuppression step (i.e. to prevent rejection of the transplanted myoblasts). Such an adequate immunosuppression can be a treatment with Tacrolimus (Kinoshita et al. 1994, 1996). Adequate immunosuppression may also be obtained by the administration to the patients of other drugs such as cyclosporine, mycophenolate mofetil or monoclonal antibodies directed against lymphocytes or proteins involved in the interactions of lymphocytes with their target cells. For examples, antibodies against CD4, CD8, ICAM-1 and LFA-1 have been shown to have immunosuppressive effects. A combination of the previous drugs alone or with antibodies may also provide adequate immunosuppression for the transplantation of the preconditioned myoblasts.

In a particular embodiment of the present invention the myoblasts to be transplanted are myoblasts having been transfected with an expression vector which express recombinant bFGF and have been preconditioned by this recombinant bFGF prior to transplantation of both myoblasts and bFGF.

### DESCRIPTION OF PREFERRED EMBODIMENTS

Although the present trend on research for the treatment of degenerative diseases involving muscle cells such as DMD seems to be towards gene therapy, rather than cell therapy, there is still a great deal of work to be done in animal models before either approach, or a mixture of both approaches will be required for the treatment of inherited myopathies such as DMD^{32,34}.

No satisfactory level of dystrophin expression was obtained following myoblast transplantation not only in clinical trials but also in animal experiments not using irradiation³³ combined with marcaine or notexin destruction of the muscle^{26,27}. These techniques are, however, too damaging, too invasive or too risky to be used in clinical trials. Basic FGF has been reported to both stimulate proliferation and inhibit differentiation of skeletal myoblasts by suppressing muscle regulatory factors such as MyoD and myogenin^{12,41}. Expression of bFGF has been examined in regenerating skeletal muscles by immunohistochemistry and in situ hybridization, and found to be up-regulated compared to non-injured muscles^{3,11}. Increased skeletal muscle mitogens have also been observed in homogenates of regenerating muscles of mdx mice³. There are increased levels of bFGF in extracellular matrix of mdx skeletal muscles¹³, mdx satellite cells associated with repair³ and such cells respond more sensitively to exogenous addition of bFGF¹⁴. There is a high degree of homology between bFGF from various species² therefore recombinant human bFGF is active on mouse cells⁹. It is then contemplated that bFGF has the same effect on myoblasts of other species, namely human. In the present series of experiments, myoblasts were pretreated with recombinant human bFGF to increase their proliferation and to verify whether such treatment which is less invasive could have beneficial effects on myoblast transplantation.

Furthermore, based on the significant improvements in the fusion properties of the preconditioned myoblasts of the present invention, a combination of gene therapy and cell therapy can be envisaged. Indeed, recombinant bFGF, derivatives or portions thereof retaining their biological activity of enhancing the fusion properties of myoblasts, can be expressed by the myoblasts to the transplanted. The means to introduce a nucleic acid encoding recombinant bFGF into a myoblast are well known in the art. Expression vectors enabling the expression of proteins are also well known in the art.

In our experiments, primary myoblast cultures from the same donors were grown with or without bFGF and transplanted simultaneously to both tibialis anterior (TA) muscles of the same mice. This seems to be a good model to verify the effect of bFGF because the same primary myoblast cultures, the same grafting conditions and the same immunosuppressive state were used. Comparing both TA muscles, in all treated mdx mice, the percentage of β-galactosidase-positive fibers (this enzyme being a reporter gene) were significantly higher in left TA muscles cultures (with bFGF) than in right TA muscles cultures (without bFGF). In the muscles grafted with myoblasts grown with bFGF, the average percentage of hybrid fibers was 34.4%, with two muscles containing over 40% of donor or hybrid fibers. These are the best results ever reported following myoblast transplantation without notexin or irradiation treatment.

In the present study, myoblasts were incubated with bFGF during 48 hours and about 5 millions of these cells (about 1.75 million myogenic cells) were injected in one TA muscle. The same number of myoblasts not incubated with bFGF was injected in the control contralateral TA muscle. The higher percentage of β-galactosidase/dystrophin-positive fibers was therefore not the consequence of a higher proliferation of the myoblasts in vitro before the transplantations.

Our in vitro results indicate that an incubation during 2 days with bFGF did not significantly modify the total number of cells and the percentage of myogenic nuclei. Basic FGF did, however, significantly inhibit the fusion of myoblasts in vitro. This resulted in a small but significant increase (35%) of the percentage of myoblasts among mononuclear cells. This increase seems too small to account alone for the more than four fold increase of effectiveness of myoblast transplantation produced by bFGF. Recently both Partridge⁷ and Karpati's²⁴ group reported that a high percentage (up to 99% in Partridge's results) of the myoblasts injected in a mouse die within 5 days. This dramatic result does not seem attributable to immunological problems since it was observed following autotransplantation²⁴ or transplantation in nude mice⁷. In our experiments, although there were slightly more cells surviving three days post-transplantation for the cultures treated with bFGF, the difference did not reach a significant level and does not seem to account alone for the 4 fold beneficial effect observed 30 days post transplantation.

Basic FGF is thought to regulate myogenesis during muscle development and regeneration in vivo³. The increase percentage of muscle fibers containing the donor gene produced by the addition of bFGF may seem surprising since bFGF was reported to inhibit differentiation of myoblasts in vitro^{1,13}. Basic FGF is, however, one of many growth factors which are liberated following muscle damage⁷. These factors, all together, certainly increase myoblast proliferation and eventually muscle repairs. We have also observed that following a two day incubation with bFGF of primary myoblast cultures, myoblast fusion occurred within a few days after removal of bFGF (data not shown). The inhibition by bFGF on myoblast fusion is therefore not irreversible. Basic FGF is already at an increased level in mdx muscle, therefore it is not surprising that direct intramuscular injection did not increase the fusion of the donor myoblasts with the host fibers. In fact, bFGF injected directly in the muscle probably stimulates the proliferation of the host as well as the donor myoblasts and therefore do not favour the donor myoblasts. On the contrary, preliminary stimulation by bFGF of the donor myoblasts in culture may favour these myoblasts to proliferate more and eventually participate more to muscle regeneration than the host myoblasts. Though bFGF stimulates the fibroblasts, which an inconvenience for primary myoblast cultures, incubation of myoblast primary culture during only 48 hours with bFGF did not adversely affect our transplantation results and did on the contrary improve them. If primary myoblast cultures were made fibroblast-free by sub-cloning, it is envisageable to precondition the donors' myoblasts for a longer time and increasing this way the number of cells to be transplanted from a relatively small biopsy.

Although the results obtained following transplantation of myoblasts grown with bFGF are not as good than those obtained using irradiation and notexin²⁷, these results are nevertheless important because no technique to destroy the muscles was used. The proposed in vitro preconditioning method might therefore be used in complete replacement of such in vivo damaging pretreatment of recipient cells, or at least in partial replacement thereof, which will result in a substantial diminution of undesirable effects.

The effects of many growth factors and trophic factors on myoblast culture have been reported, it is possible that other factors such as insulin growth factor I, transferrin, platelet-derived growth factor, epidermal growth factor, adrenocorticotrophin and macrophage colony-stimulating factor may also have similar or even better effects than bFGF on the success of myoblast transplantation⁷. Furthermore, since the effect of bFGF is mediated by proteins kinase C, pharmacological agents used to enhance the activity of these enzymes (like phorbol esters) or mimicking the effect thereof (agonists) might also be used for preconditioning myoblasts. Therefore, at least one of these factors can be used alone or in combination with or without bFGF to enhance the success of myoblast transplantation. While the mechanism involved remains speculative, bFGF seems to improve the long term viability, multiplication and fusion of myoblasts. Our results suggest that pretreatment of myoblasts with bFGF may be one procedure that may increase the success of myoblast transplantation in patients suffering from a degeneration of muscle tissue and more particularly of DMD patients.

### General Definitions

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (i.e. genomic DNA, cDNA) and RNA molecules (i.e. mRNA). The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (i.e. DNA or RNA) for practicing the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hydrizidation thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “oligonucleotide” or “DNA” molecule or sequence refers to a molecule comprised of the deoxyribonucleotides adenine (A), guanine (G), thymine (T) and/or cytosine (C), in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5×Denhardt's solution, 1% SDS, and 100 μg/ml denatured carried DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein. It will be readily recognized by the person of ordinary skill, that the nucleic acid sequence of the present invention can be incorporated into anyone of numerous established kit formats which are well known in the art.

A “heterologous” (i.e. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often refered to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (i.e. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boses and “CCAT” boxes. Prokaryotic promoters contain Shine-Dalgarno sequences in addition to the −10 and −35 consensus sequences.

As used herein, the designation “functional derivative” denotes, in the context of a functional derivative of a sequence whether an nucleic acid or amino acid sequence, a molecule that retains a biological activity (either function or structural) that is substantially similar to that of the original sequence (i.e. enhances the muscle fusion properties of myoblasts). This functional derivative or equivalent may be a natural derivatives or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology. all these methods are well known in the art.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are examplified in Remington's Pharmaceutical Sciences (1980). Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in all other cellular components.

As used herein, the terms “molecule”, “compound”, “agent”, or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The agents can be selected and screened by a variety of means including random screening, rational selection and by rational design using for example protein or ligand modelling methods such as computer modelling. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modelling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in enhancing the fusion-enhancing properties of bFGF.

In one embodiment, bFGF may be provided as a fusion protein. The design of constructs therefor and the expression and production of fusion proteins are well known in the art (Sambrook et al., 1989, supra; and Ausubel et al., 1994, supra). Non limiting examples of such fusion proteins include a hemaglutinin fusions and Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

Although bFGF contains its own signal sequence, in certain embodiments, it might also be beneficial to fuse the sequence of bFGF encoding the muscle fusion-enhancing property of the present invention to heterologous signal peptide sequences enabling a secretion of the fusion protein from the host cell. Signal peptides from diverse organisms are well known in the art. Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) myoblast muscle fusion-enhancing polypeptide. It will be clear to the person of ordinary skill that whether a bFGF polypeptide of the present invention, variant, derivative, or fragment thereof retains its function in preconditioning myoblasts can be readily determined by using the teachings and assays of the present invention and the general teachings of the art.

As exemplified herein below, the interaction domains of the present invention can be modified, for example by in vitro mutagenesis, to dissect the structure-function relationship thereof and permit a better design and identification of modulating compounds. A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, an advantage might be rendered moot if both polypeptide tested directly interact. It will be understood that extracts from mammalian cells for example could be used in certain embodiments, to compensate for the lack of certain factors.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, erythropoietic cells can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a liposome, which can be designed to be targeted to a specific cell type, and engineered to be administered through different routes.

For administration to humans, the prescribing medical professional will ultimately determine the appropriate form and dosage for a given patient, and this can be expected to vary according to the chosen therapeutic regimen (i.e. DNA construct, protein, cells), the response and condition of the patient as well as the severity of the disease.

Composition within the scope of the present invention should contain the active agent (i.e. fusion protein, nucleic acid, and molecule) in an amount effective to achieve the desired therapeutic effect while avoiding adverse side effects. Pharmaceutically acceptable preparations and salts of the active agent are within the scope of the present invention and are well known in the art (Remington's Pharmaceutical Science, 16th Ed., Mack Ed.).

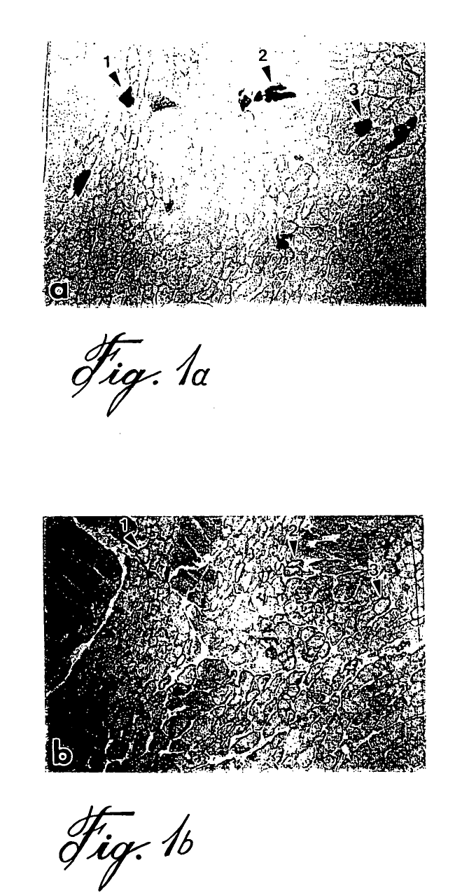

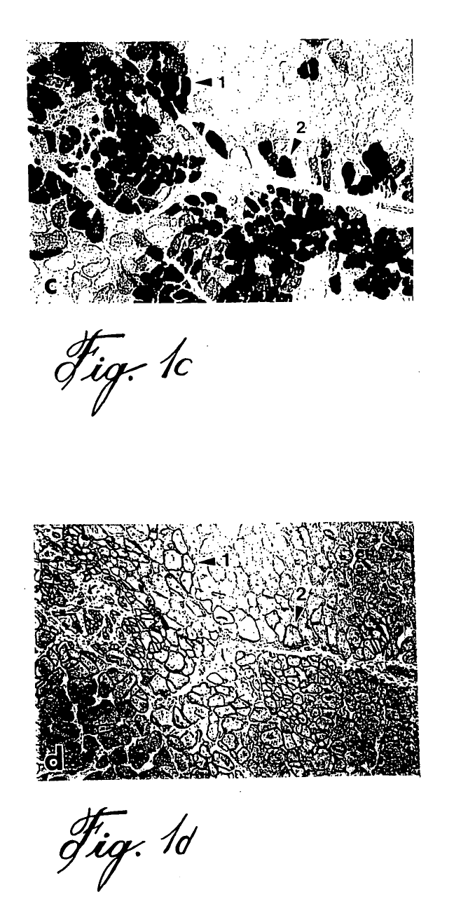

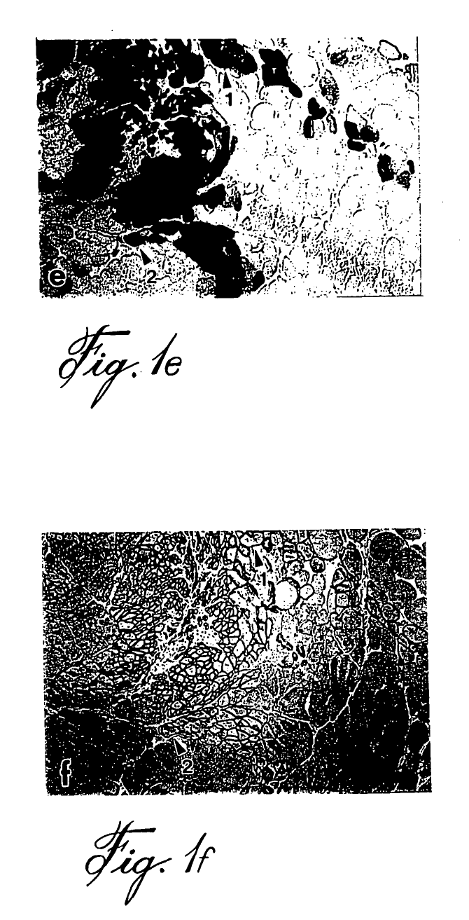

The present invention will be further described by way of the following Examples and FIG. 1, which purpose is to illustrate this invention rather than to limit its scope.

### BRIEF DESCRIPTION OF FIG. 1

This FIGURE shows cross sections of TA muscle of mdx mice 28 days after injection of the transgenic myoblasts. Pairs of serial sections from 3 different muscles of three mice are illustrated. Panels a and b illustrate sections of muscles injected with myoblasts grown without bFGF. Panels c to f illustrate sections of muscles injected with myoblasts grown with bFGF. In each pair, one section was stained for β-galactosidase (panels a, c and e). The other section of the pair was immunostained for dystrophin (panels b, d and f).

The muscles injected with myoblasts grown in presence of bFGF contained much more β-galactosidase and dystrophin positive fibers than muscles injected with myoblasts grown without bFGF. Most muscle fibers expressing β-galactosidase were dystrophin-positive. In each pair of panels, the same muscle fibers are identified by the same numbers. Scale bar is 100 μm.

### EXAMPLE 1

### Enhancement of the Muscle Fusion Properties of Myoblasts by the Action of bFGF

Materials and Methods

Myoblast Cultures

Primary myoblast cultures were established from muscle biopsies of newborn transgenic mice²⁶. The founder mouse (TnI Lac Z1/29) was provided by Dr. Hasting (McGill University, Montreal, Canada) onto the CD1 background and was reproduced in our laboratory. This transgenic mouse expresses the β-galactosidase gene under the control of the promoter of the quail fast skeletal muscle troponin I gene¹⁶. Blue muscle fibers are revealed in these transgenic mice following incubation with a substrate, 5-brom-4-chlor-3-indolyl-β-D-galactopyronoside (X-gal) (Boehringer Mannheim Canada, Laval, Canada). Before starting myoblast cultures, it was necessary to identify transgenic newborns by X-gal staining of a small muscle biopsy because heterozygote transgenic mice were used as parents. Myogenic cells were released from skeletal muscle fragments of the transgenic newborns by serial enzyme treatments. First, a one hour digestion was done with 600 U/ml collagenase (Sigma, St-Louis, Mo., USA). This was followed by a 30 minute incubation in Hanck's balanced salt solution (HBSS) containing 0.1% w/v trypsin (Gibco Lab, Grand Island, N.Y., USA). Satellite cells were placed in 75 cm² culture flasks (Coster, Cambridge, Mass., USA) in proliferating medium, i.e. 199 medium (Gibco Lab.) with 15% fetal bovine serum (Gibco Lab.), 1% penicillin (10,000 U/ml) and 1% streptomycin (10,000 U/ml).

Myoblast Transplantation

One day after starting culture, the culture medium of some flasks was replaced by medium containing 100 ng/ml human recombinant bFGF (Sigma). Three days after starting culture, myoblasts were detached from the flasks with 0.1% trypsin followed by three suspensions in HBSS and centrifugations (6500 RPM, 5 minutes). The final cell pellet was diluted in only 40 μl of HBSS.

Seventeen C57BU10ScSn mdx/mdx mice (mdx mice) approximately one month old were used for this experiment. This work was authorized and supervised by the Laval University Animal Care Committee and was conducted according to the guidelines set out by the Canadian Council of Animal Care.

The mdx mice were divided in three groups. Six mdx mice of one group were grafted in both tibialis anterior (TA) muscles: myoblasts grown with bFGF were injected in the left TA and myoblasts grown without bFGF were injected in the right TA. Myoblasts grown without bFGF were injected in only the left TA of six other mdx mice. These six mdx mice were then injected intramuscularly four times (after grafting 0, +1, +4 and +6 days) either with 10 μl of bFGF (100 ng/ml, 3 mice) or with 10 μl of HBSS (3 mice). The last five mice were grafted in both TA muscle with normal CD1 mouse myoblasts infected with replication defective retroviral vector LNPOZC7 (gift from Dr C. Cepko, Harward, Boston, Mass.) which contains the LacZ gene. The left TA muscles were injected with 4 million myoblasts grown with bFGF, while the right TA muscles were injected with 4 million myoblasts grown without bFGF. Three days after grafting, these 5 mice were sacrificed to detect the number of β-galactosidase positive cells which survived in each TA muscle. The numbers of β-galactosidase positive cells were counted in 8 μm sections obtained at every 160 μm throughout the muscle. The total number of cells counted was multiplied by 20 to obtain an estimate of the number of surviving cells and a correction was made to account for the percentage of unlabelled cells in cultures with and without bFGF.

For the myoblast injection, the mice were anesthetized with 0.05 ml of a solution containing 10 mg/ml of ketamine and 10 mg/ml xylazine. The skin was opened to expose the TA muscle. The myoblast suspension was taken up into a glass micropipette with 50 μm tip (Drummond Scientific Company, Broomall, Pa., USA). The TA muscle was injected at 10 sites with a total of about 5 million cells. The skin was then closed with fine sutures. FK 506 (Fujisawa Pharmaceutical Co Ltd, Osaka, Japan) was administered at 2.5 mg/kg to immunosuppress the animals. Alternatively, the immunosuppressive treatment can be made by other pharmacological agents like cyclosporin (Sandoz), RS61443 (Syntex) or rapamycin (Wyeth-Ayerst)⁴².

Muscle Examination

Three or twenty-eight days after myoblast transplantation, the mice were sacrificed by intracardiac perfusion with 0.9% saline under deep anesthesia of 10 mg/ml ketamine and 10 mg/ml xylazine. The TA muscles were taken out and immersed in a 30% sucrose solution at 4° C. for 12 hours. The specimens were embedded in OCT (Miles Inc, Elkhart, Ind., USA) and frozen in liquid nitrogen. Serial cryostat sections (8 μm) of the muscles were thawed on gelatin coated slides. These sections were fixed in 0.25% glutaraldehyde and stained in 0.4 mM X-gal in a dark box overnight (12 hours) at room temperature to detect the muscle fibers containing β-galactosidase. Dystrophin was detected on adjacent cryostat sections by an immunoperoxidase technique with a sheep polyclonal antibody against the 60 KD dystrophin fragment (R27, Genica Co, Boston, Mass., USA) and the peroxidase activity was revealed by a 10 minute incubation with 3,3′ diaminobenzidine (DAB, 0.5 mg/ml, Sigma) and hydrogen peroxidase (0.015%).

Desmin Staining

The primary cultures were washed with PBS and fixed with 100% methanol at −4° C. They were then washed again 3 times with PBS and incubated 1 hr with a mAb anti-human desmin (Dako, Copenhagen, Denmark) diluted 1/50 with PBS containing 1% blocking serum (i.e. 0.33% rabbit serum, 0.33% horse serum and 0.33 fetal calf serum). They were washed 3 times with PBS with 1% blocking serum and incubated 1 hr with a 1/100 dilution (in PBS with 1% blocking serum) of a rabbit anti-mouse immunoglobulin (Dako). Following 3 washes with PBS, the peroxidase activity was revealed with DAB as for dystrophin immunohistochemistry.

Results

Myoblasts from muscle biopsies of transgenic mice expressing β-galactosidase under a muscle specific promoter were grown with or without bFGF and injected in mdx muscles not previous irradiated or damaged with notexin. A month later, the animals were sacrificed and the injected muscles were examined for the presence of β-galactosidase and dystrophin. Many positive muscle fibers were observed. In our previous experiments, muscles of mdx mice which did not receive injections of transgenic myoblasts remained completely devoid of β-galactosidase-positive fibers²². Therefore all β-galactosidase-positive muscle fibers observed in grafted mdx muscles are resulting from the fusion of some donor myoblasts among themselves (donor's fibers) or with the host myoblasts (hybrid fibers). In serial muscle sections, most of the β-galactosidase-positive muscle fibers were observed to be also dystrophin-positive (FIG. 1). In all biopsied TA muscles, the number of β-galactosidase-positive muscle fibers was counted and expressed as a percentage of the total number of fibers in a cross section. The sections containing of the maximum percentage of β-galactosidase-positive muscle fibers were selected for each muscle. In mdx mice grated in both TA muscles, the percentage of β-galactosidase-positive muscle fibers in the left TA muscle (grafted with myoblasts grown with bFGF) was compared with that in the right TA muscle (grafted with myoblasts grown without bFGF) of the same mouse (Table 1). Without notexin and irradiation, only a low percentage of hybrid or donor muscle fibers were observed in the right TA muscle i.e. the mean number of β-galactosidase-positive fibers per muscle cross section was 156.3 giving a mean percentage of β-galactosidase-positive fibers of 8.396. The left TA muscles contained, however, significantly more hybrid or donor muscle fibers, i.e. the mean number of β-galactosidase-positive fibers per muscle cross section was 773.7 thus giving a mean percentage of β-galactosidase-positive fibers equal to 34.4% (FIG. 1). This is more than a four fold increase in the efficacy of myoblast transplantation produced by the addition of bFGF to the culture medium.

We have also investigated whether the beneficial effect of bFGF could be obtained by injecting it directly in the muscle at 4 intervals after myoblast transplantation. No significant difference in the percentage of hybrid or donor muscle fibers (i.e. β-galactosidase positive fibers) was observed between the groups which received intramuscular injections of bFGF and those which received HBSS injections (control) (Table 2). The percentage of β-galactosidase positive muscle fibers was, however, higher following repeated injection of HBSS (14.8%) or of bFGF (15.9%) than following injection of myoblasts alone grown without bFGF (Table 1, 8.3%). This may be due to damage produced by the repeated injections which may increase the regeneration process.

It has been reported recently by Huard et al.²¹ and by Beauchamp et al.⁷, that a high percentage of the myoblasts injected in a muscle died within the first few days following their transplantation. To examine whether the increase efficiency of myoblast transplantation following culture with bFGF could be due to a reduced cell death, we have labelled normal CD1 primary cultures grown with or without bFGF with a retroviral vector containing the β-galactosidase gene under an LTR promoter. Normal myoblasts were labelled with a retroviral expressing β-galactosidase because only mature myoblasts and myotubes of transgenic TnI LacZ 1/29 can express β-galactosidase. With labelling using a retroviral vector a higher percentage of the cells in the primary culture expressed the reporter gene. The retrovirally labelled cells were then injected in a muscle of 5 mice. We examined the number of β-galactosidase positive cells 3 days after their transplantation. In all 5 mice, the number of the cells was not significantly higher in left TA muscles (with bFGF) (3.29±1.54×10⁵ cells) than in right TA muscles (without bFGF 2.13±0.40×10⁵ cells). Note that since 4×10⁶ cells were injected in each muscle, there is only 5.3% of the injected cells surviving at 3 days without bFGF while only 8.2% of the injected cells survived with bFGF.

To try to understand the beneficial effects of bFGF on myoblast transplantation, we examined the effect of a short stimulation (2 days) with 100 ng/ml bFGF on primary myoblast cultures. The total number of cells, in each flask was not significant different (31.9±6.8×10⁶ with bFGF n=5, 30.0±5.8×10⁶ without FGF n=9, unpaired t-test: p=0.573). The myoblasts and myotubes were then identified by revealing desmin by immunoperoxidase. In these cultures, there was no difference in the percentage of myogenic nuclei (nuclei in myoblasts and in myotubes) between the two groups of cultures (Table 3, line 1). More myogenic cells were however fused in the absence of bFGF (Table 3, line 2). There was an higher percentage of the total nuclei (including myoblasts, myotubes and fibroblasts) which were myoblast nuclei in cultures containing bFGF (Table 3, line 3). The increase of myoblasts was more clear when the percentage of myoblasts was calculated among mononuclear cells (excluding the myotubes) (Table 3, lines 4 and 5). This was however only a 35% increase.

TABLE 1
Effect of culture with or without bFGF on the formation of
muscle fibers containing donor's gene in mdx mice

| | no bFGF (right TA | with bFGF (left TA |
|---|---|---|
| | muscle) | muscle) |
| No of | No (%) of β-gal. | No (%) of β-gal. |
| mdx mice | positive fibers | positive fibers |
| 1 | 170(11.0) | 514(33.3) |
| 2 | 259(11.9) | 438(20.4) |
| 3 | 259(13.1) | 1007(37.4) |
| 4 | 57(4.1) | 695(34.0) |
| 5 | 139(6.1) | 848(43.8) |
| 6 | 54(3.6) | 1140(51.7) |
| Mean ± SD | 156.3 ± 91.5(8.3 ± 4.2)# | 773.7 ± 275.8(34.4 ± 12.8)# |

#Paired t-test indicated a significant difference (p < 0.05)

TABLE 2
Effect of intramuscular injections of bFGF in mdx mice

| | No (5%) of β-gal. | |
|---|---|---|
| | positive fibers | Mean ± SD |
| HBSS IM | | |
| injections | | |
| 1 | 180(12.4) | 372.0 ± 172.8(14.8 ± 2.9) |
| 2 | 421(14.1) | |
| 3 | 515(18.0) | |
| bFGF IM | | |
| injections | | |
| 1 | 176(7.4) | 289.7 ± 167.5(15.9 ± 8.4) |
| 2 | 482(24.1) | T test indicated no |
| 3 | 211(16.3) | significant difference |
| | | (p > .05) |

TABLE 3
Effects of bFGF on primary myoblast culture

| | no bFGF | with bFGF | |
|---|---|---|---|
| | (mean ± SD) | (mean ± SD) | sign |
| 1) % of myoblast and myotube | 34.5 ± 5.3 | 35.1 ± 4.8 | 0.81 |
| nucleic relative to total nuclei | | | |
| 2) % of myotube nuclei relative | 40.8 ± 8.0 | 11.5 ± 6.6 | 0.0001 |
| to total myotube and | | | |
| myoblast nuclei | | | |
| 3) % myoblast nuclei relative to | 21.1 ± 3.6 | 30.9 ± 3.8 | 0.0001 |
| total nuclei | | | |
| 4) % myoblast nuclei relative to | 23.9 ± 5.4 | 32.2 ± 4.1 | 0.001 |
| non myotube nuclei | | | |
| 5) % of non-myoblast nuclei | 76.1 ± 5.4 | 67.8 ± 4.1 | 0.001 |
| relative to non myotube nuclei | | | |

### EXAMPLE 2

### Treatment of Patients Suffering of Muscular Distrophy with Pretreated Myoblasts

The above results can be extrapolated to an in vivo utility and verified in patients suffering of muscular dystrophy. The healthy donors and DMD recipients should be matched, if possible, upon their compatibility for the MHC (HLA)-class I (A,B,C) and -class II (Dr) antigens. The dystrophic patients should undertake an immunosuppressive treatment by being administered, for example, FK 506, cyclosporin, RS61443 or rapamycin. Donors' biopsy would then be treated substantially in accordance with the procedures given in Example 1 with regard to mice myoblasts. The success of the transplantation might be monitored by measuring the incidence of dystrophin-positive fibers from a biopsy obtained from the site of transplantation and by evaluating the resulting increase of muscular strength³⁹.

### EXAMPLE 3

### Compositions Comprising Preconditioned Myoblasts to Enhance Their Muscle Fusion Properties

The present invention thus also provides compositions comprising preconditioned myoblasts which are ready to be injected in the muscles of a patient in need of said myoblast injection (or of an animal model system). These myoblasts are preconditioned to improve their survival, proliferation in vivo and eventual fusion with the existing muscle fibers and in a particular embodiment to introduce heterologous genes.

The preconditioning of the myoblasts includes growth thereof in a culture medium which comprises at least basic fibroblast growth factor (bFGF) but which may also include other growth factors such as insulin growth factor I (IGF-1), transferrin, platelet derived growth factor (PDGF), epidermal growth factor (EGF), adrenocorticotrophin macrophage colony-stimulating factor, protein kinase C activators and any combination thereof.

Of course, it will be clear to the skilled artisan that this composition should be exempt from infection agents such as viral agents. Non limiting examples of viral agents include HIV, hepatitis B and C, CMV. Tests which enable detection of such infections agents are well known in the art. The composition will also be tested and certified to be free of bacterial and mycoplast infections. Such tests are also well known in the art. Preferably, the composition should be certified as exempt of endotoxins.

The myogenicity of the cellular composition will be previously tested in vitro and in vivo and certified. The test of myogenicity in vitro will be based on the culture of a sample of the product in conditions favoring the fusion of myogenic cells. The conditions comprising a low serum concentration and the absence of growth factors, either promoting the proliferation or inhibiting the fusion. The in vivo myogenicity testing of the cellular composition will be based on the transplantation of a sample of the product in an immunodeficient mouse. Such immunodeficient mouse being for example SCID mouse, SCID-Bg mouse or in a genetically modified mouse which has been made immunodeficient, for example Rag mouse).

The cellular composition will also be tested for tumorigenicity. This again will involve two types of tests, i.e., in vitro test and in vivo test. The in vitro test is based on the absence of proliferation of a sample of the nontumorigenic cellular composition in soft agar, which the tumorigenic cells will proliferate in such a condition. The in vivo test will verify the absence of a tumor following the transplantation of a sample of the product in SCID, SCID-Bg or other genetically modified mice which are immunodeficient, such as the Rag mouse.

The cellular composition will also be tested to confirm that the preconditioned cells are indeed from the specific donor of a given patient. This confirmation of the donor identity will be carried out by DNA testing as commonly known in the art. This testing includes a confirmation of the presence of the same DNA polymorphisms in the cellular product as in the blood cells of the donor. The polymorphisms used for identification will include test for VNTR (Variable Number of Tandem Repeats) or micro satellite markers. Other tests of DNA polymorphisms may also fulfill the aim of certifying the origin of the cellular composition.

The cellular product will be delivered in a ready to inject formula containing Hank's Balanced Salt Solution (HBSS). The cells will be concentrated at 150 millions per mL. However, other cellular concentrations and compositions of the injection medium may be found more adequate for other types of applications.

In a preferred embodiment, the composition according to the present invention include myoblasts which are preconditioned to survive, proliferate and fuse with muscle fibers following injection in a muscle. This product is certified of donor origin and is certified as non infectious, non tumorigenic, fusion competent and exempt of endotoxins.

In a particular embodiment, the bFGF cDNA is introduced in a retroviral expression vector such that it is under the control of a strong promoter such as the SV40 or CMV promoters. These strong promoters allow the expression of bFGF gene to be high enough to produce the muscle fusion promoting amount of bFGF (between 10 ng and 1 μg per ml). The patent publication WO99/30730 shows that such promoters were capable of achieve amounts of gene products of the order of hundreds nanograms per day per 10⁶ cells (see page 8, lines 1 to 3). These promoters would therefore easily succeed in achieving the expected amounts of bFGF in the culture of myoblasts. The levels of bFGF can be easily monitored since they are produced in vitro in the culture medium. The genetic engineering methods required for such a construction are well known in the art. The presence of selectable marker in the retroviral vector (i.e. hygromycin) enables a positive selection of the transfected myoblasts. The genetically engineered myoblasts can now be cultured to achieve the desired confluency and used to assess the fusion enhancing properties of the bFGF expressed thereby. Such genetically engineered myoblasts will be tested in accordance With the methods of the present invention to assess the effect of recombinantly expressed bFGF (i.e. Examples 1-2). Myoblasts having been transfected with the same retroviral vector without an insert will serve as a control.

### REFERENCES

1. Allen R E and Boxhorn A L: Regulation of skeletal muscle satellite cell proliferation and differentiation by transforming growth factor-beta, insulin-like growth factor 1, and fibroblast growth factor. J Cell Physiol 1989;138: 311-315.
2. Anderson J E, Kakulas B A, Jacobsen P F, Johnsen R D, Kornegay J N and Grounds M D: Comparison of basic growth factor in x-linked dystrophin-deficient myopathies of human, dog and mouse. Growth Factor 1993; 9: 107121.
3. Anderson J E, Liu L and Kardami E: Distinctive patterns of basic fibroblast growth factor (bFGF) distribution in degenerating and regenerating areas of dystrophic (mdx) striated muscles. Develop Biol 1991;147: 96-109.
4. Austin L, Bower J, Kurek J and Vakakis N: Effect of leukaemia inhibitory factor and other cytokines on murine and human myoblast proliferation. J Neurol Sci 1992;112:185-191.
5. Austin L and Burgess A W: Stimulation of myoblast proliferation in culture by leukaemia inhibitory factor and other cytokines. J Neurol Sci 1991;101: 193-197.
6. Arahata K, Ishiura S, Tsukahara T, Suhara Y, Eguchi C, Ishihara T, Nonaka I, Ozawa E and Sugita H: Immunostaining of skeletal and cardiac muscle membrane with antibody against Duchenne muscular dystrophy peptide. Nature 1988; 333: 861-863.
7. Beauchamp J R, Morgan J E, Pagel C N, Partridge T A: Quantitative studies of the efficacy of myoblast transplantation. Muscle and Nerve Supp. 1, S261, 1994.
8. Bischoff. R: A satellite cell mitogen from crushed muscle. Dev. Biol. 1986; 115:140-147.
9. Chen G and Quinn L S: Partial characterization of skeletal myoblast mitogen in mouse crushed muscle extract. J Cell Physiol 1992;153: 563-574.
10. Chen M, Li H J, Fang Q, Goodwin T G, Florendo J A and Law P K: Dystrophin cytochemistry in mdx mouse muscle injected with labeled normal myoblasts. Cell Transpl 1992;1: 17-22.
11. Clarke M S F, Khakee R and McNeil P L: Loss of cytoplasmic basic fibroblast growth factor from physiologically wounded myofibers of normal and dystrophic muscle. J Cell Sci 1993;106: 121-133.
12. Clegg C H, Linkhart T A, Olwin B B and Hauschka S D: Growth factor control of skeletal muscle differentiation: Commitment to terminal differentiation occurs in G1 phase and repressed by fibroblast growth factor. J Cell Biol 1987; 105: 949-956.
13. DiMario J, Buffinger N, Yamada S and Strohman R C: Fibroblast growth factor in the extracellular matrix of dystrophic (mdx) mouse muscle. Science 1989; 244: 688-690.
14. DiMario J and Strohman R C: Satellite cells from dystrophic (mdx) mouse muscle are stimulated by fibroblast growth factor in vitro. Differentiation 1988; 39: 42-49.
15. Florini J R and Margi K A: Effect of growth factors on myogenic differentiation. Am J Physiol 1989; 256: C701-C711.
16. Grounds M D: Towards understanding skeletal muscle regeneration. Path Res Pract 1991;187:1-22.
17. Gussoni E, Pavlath P K, Lanctot A M, Sharma K, Miller R G, Steinman L and Blaud H M: Normal dystrophin transcripts detected in DMD patients after myoblast transplantation. Nature 1992; 356: 435-438.
18. Hallauer S M, Bradshaw H W and Hasting K E M: Complex fiber-type specific expression of fast skeletal muscle troponin I gene constructs in transgenic mice. Development 1993;119: 691-701.
19. Hoffman E P, Brown R H and Kunkel L M: Dystrophin: the protein product of Duchenne muscular dystrophy locus. Cell 1987; 51: 919-928.
20. Hsu et al.: J. Biol. Chem. 1994; 25: 9213.
21. Huard J, Ascadi G, Jani A, Massi B: Gene transfer into mdx skeletal muscle by isogenic, genetically labelled myoblasts. Muscle and Nerve, Suppl. 1, 5260, 1994.
22. Huard J, Bouchard J P, Roy R, Malouin F, Dansereau G, Labrecque C, Albert N, Richards C L, Lemieux B and Tremblay J P: Human myoblast transplantation: preliminary results of 4 cases. Muscle and Nerve 1992; 15: 550-560.
23. Huard J, Verreault S, Roy R, Tremblay M and Tremblay J P: High efficiency of muscle regeneration following human myoblast clone transplantation in SCID mice. J Clin Invest 1994; 93: 586-599.
24. Karpati G, Ajdukovic D, Arnold D, Gledhill R B, Guttmann R, Holland P, Koch P A, Shoubridge E, Spence D, Vanasse M, Watters G V, Abrahamowicz M, Duff C and Worton R G: Myoblast transfer in Duchenne muscular dystrophy. Ann Neurol 1993; 34: 8-17.
25. Karpati G, Pouliot Y, Zubrzyccka-Gaarn E, Carpenter S, Ray P N, Worton R G and Holl P: Dystrophin is expressed in mdx skeletal muscle fibers after normal myoblast implantation. Am J Pathol 1989; 135: 27-32.
26. Kinoshita I, Huard J and Tremblay J P: Utilization of myoblasts from transgenic mice to evaluate the efficacy of myoblast transplantation. Muscle and Nerve 1994;17:975-980.
27. Kinoshita I, Vilquin J P, Guerette B, Asselin I, Roy R, Tremblay J P. Very efficient myoblast allotransplantation in mice under FK506 immunosuppression. Muscle and Nerve, 1994; 17: 1407-1415.
28. Kinoshita, I., Roy, R., Dugre, F. J., Gravel, C., Vilquin, J. T., Roy, B., Goulet, M., Asselin, I. and Tremblay, J. P. Myoblast transplantation in monkeys: control of immune response by FK506. J. Neuropath and Exp. Neurol. 55: 687-697, 1996.
29. Koenig M, Hoffman E P, Bertelson C J, Monaco A P, Feener C and Kunkel L M: Complete cloning of the Duchenne muscular dystrophy (DMD) cDNA and preliminary genomic organization of the DMD gene in normal and affected individuals. Cell 1987; 51: 919-928.
30. Labrecque C, Roy R and Tremblay J P: Immune reaction after myoblast transplantation in mouse muscle. Transplant Proc 1992; 24: 2889-2892.
31. Law P K, Bertorini T E, Goodwin T G, Chen M, Fang Q, Kirby D S, Florendo J A, Herrod H G and Golden G S: Dystrophin production induced by myoblast transfer therapy in Duchenne muscular dystrophy. Lancet 1990; 336: 114115.
32. Lowe et al. Endocrinology 1993; 132: 1593.
33. Morgan J E: Cell and gene therapy in Duchenne muscular dystrophy. Human Gene Therapy 1994; 5:165-173.
34. Morgan J E, Pagel C N, Sherratt T and Partridge T: Long-term persistence and migration of myogenic cells injected into preirradiated muscles of mdx mice. J Neurol Sci 1993;115:191-200.
35. Partridge T A: Myoblast transfer: a possible therapy for inherited myopathies? Muscle and Nerve 1991;14:197-212.
36. Partridge T A, Morgan J E, Coulton G R, Hoffman E P and Kunkel L M: Conversion of mdx myofibers from dystrophin negative to positive by injection of normal myoblasts. Nature 1989; 337: 176-179.
37. Patte and Blanquet. Cell. Mol. Biol. 1992; 38: 429.
38. Sugita H, Arahata K, Ishiguro T, Suhara Y, Tsukahara T, Ishiura S, Eguchi C, Nonaka I and Ozawa E: Negative immunostaining of Duchenne muscular dystrophy (DMD) and mdx muscle surface membrane with antibody against synthetic peptide fragment predicated from DMD cDNA. Proc Japan Acad 1988; 64: 37-39.
39. Tremblay J P, Bouchard J P, Malouin F, Theau D, Cottrell F, Collin H, Rouche A, Gilgenkrantz S, Abbadi N, Tremblay M, Tome F M S and Fardeau M: Myoblast transplantation between monozygotic twin girl carrier of Duchenne muscular dystrophy. Neuromusc. Disord. 1993; 5: 583-592.
40. Tremblay J P, Malouin F, Roy R, Huard J, Bouchard J P, Satoh A and Richards C L: Results of a triple blind clinical study of myoblast transplantations without immunosuppressive treatment in young boys with Duchenne muscular dystrophy. Cell Transplantation 1993; 2: 99-112.
41. Trenari et al. Int. J. Cancer 1994; 56: 219.
42. Vaidva T B, Rhodes S J, Traparowsky E J and Konieczny S F: Fibroblast growth factor and transforming growth factor β repress transcription of myogenic regulatory gene MyoD1. Molecular Cellular Biol 1989; 9: 3576-3579.
43. Velquin et al. Transpl. Proc. 1994; 26: 3372.
44. Wakeford S, Watt D J, Partridge T A: X-irradiation improves mdx mouse muscle as a model of myofiber loss in DMD. Muscle and Nerve 1991; 14: 4250.

1. A composition comprising a culture of myoblasts to be transplanted into a recipient muscle tissue, together with a suitable pharmaceutical carrier, said culture comprising myoblasts and a muscle-fusion promoting amount of human basic fibroblast growth factor (bFGF), which transplanted myoblasts have been grown in the presence of said amount of bFGF prior to transplantation into said recipient muscle tissue, said amount being capable of increasing by at least two fold the fusion between transplanted and recipient myoblasts over and above the fusion of a same number of transplanted myoblasts not grown in the presence of said promoting amount of bFGF.

2. The composition of claim 1 wherein said bFGF is added exogenously to said culture of myoblasts.

3. The composition of claim 1, wherein said bFGF is produced endogenously in said culture of myoblasts by genetically engineering myoblasts to express a gene sequence encoding bFGF under the control of a promoter capable of governing the production of said amount of bFGF.

4. The composition of claim 3, wherein said promoter is a viral promoter.

5. A method of improving the fusion of myoblasts upon transplantation thereof into a recipient muscular tissue, comprising the steps of:
growing a culture of myoblasts comprising myoblasts which have been genetically engineered to express human basic fibroblast growth factor (bFGF) during in vitro culturing and to produce same in said culture in an amount capable of increasing by at least two fold muscle fusion between transplanted and recipient myoblasts upon transplantation, over and above the fusion obtained with the same number of transplanted myoblasts not grown in the presence of said amount of bFGF; and
transplanting said culture of myoblasts into a recipient muscle tissue along with said amount of bFGF.

6. A method of improving the fusion of myoblasts upon transplantation thereof into a recipient muscular tissue comprising the steps of:
growing unpurified primary myoblasts in culture in the presence of an exogenously added amount of human basic fibroblast growth factor (bFGF) capable increasing by at least two fold the fusion between transplanted and recipient myoblasts upon transplantation, over and above the fusion obtained with the same number of transplanted myoblasts not grown in the presence of said amount of bFGF; and
transplanting said culture of myoblasts into a recipient muscular tissue along with said amount of bFGF.

7. A composition as defined in claim 1, wherein said culture of myoblasts comprises fibroblasts.

8. A method according to claim 5, wherein said culture of myoblasts comprises fibroblasts.

9. A method according to claim 6, wherein said culture of myoblasts comprises fibroblasts.

10. A composition according to claim 7, wherein said culture of myoblasts comprises primary myoblasts cultured for two days in the presence of bFGF.

11. A method according to claim 8, wherein said culture of myoblasts comprises primary myoblasts cultured for two days in the presence of bFGF.

12. A method according to claim 9, wherein said culture of myoblasts comprises primary myoblasts cultured for two days in the presence of bFGF.

13. A composition as defined claim 1, wherein said amount of bFGF is 100 ng bFGF per ml of composition.

14. A method as defined in claim 5, wherein said amount of bFGF is 100 ng bFGF per ml of composition.

15. A method as defined in claim 6, wherein said amount of bFGF is 100 ng bFGF per ml of composition.
